(19) United States
(12) Patent Application Publication
(10) Pub. No.: US 2025/0302934 A1
(43) Pub. Date: Oct. 2, 2025

(54) SET DOMAIN-CONTAINING 2 (SETD2) VACCINE

(71) Applicant: INSTITUTE FOR CANCER RESEARCH D/B/A THE RESEARCH INSTITUTE OF FOX CHASE CANCE CENTER, Philadelphia, PA (US)

(72) Inventors: Philip ABBOSH, Philadelphia, PA (US); Marya Kozinova, Philadelphia, PA (US); Robert N. Uzzo, Philadelphia, PA (US); Alexander Metz, Philadelphia, PA (US)

(21) Appl. No.: 18/864,271

(22) PCT Filed: May 8, 2023
(86) PCT No.: PCT/US2023/066716

§ 371 Date: Nov. 8, 2024

Related U.S. Application Data
(60) Provisional application No. 63/339,690, filed on May 9, 2022.

(51) Int. Cl. A61K 39/00; A61K 38/00; A61K 45/06; C12N 9/10; C12Q 1/6841; C12Q 1/6886

(52) U.S. Cl. CPC A61K 39/001154; A61K 45/06; C12N 9/1007; C12Q 1/6841; C12Q 1/6886; A61K 38/00; C12Q 2600/156; C12Y 201/01

(57) ABSTRACT

Pharmaceutical compositions comprising one or more peptides derived from aberrantly translated retained introns (ATaRIs), methods for treating cancer with the same, and methods of immunizing a subject against cancer or eliciting an immune response to one or more peptides derived from ATaRIs are provided herein.

### REFERENCE TO SEQUENCE LISTING

This application includes a Sequence Listing filed electronically as an XML file named 853003346SEQ, created on May 5, 2023, with a size of 10,946,423 bytes. The Sequence Listing is incorporated herein by reference.

### FIELD

The present disclosure is directed, in part, to pharmaceutical compositions comprising one or more peptides derived from Aberrantly Translated Retained Introns (ATaRIs), methods for treating cancer with the same, and methods of immunizing a subject against cancer or eliciting an immune response to one or more peptides derived from ATaRIs.

### BACKGROUND

Introns within messenger RNA (mRNA) are normally removed during their processing. However, in some cases, introns are retained, particularly when the SET Domain-Containing 2 (SETD2) gene is mutated, as it commonly in kidney cancer and other forms of cancer (e.g., liver cancer, mesothelioma, lung cancer, etc.). When SETD2 is deleteriously mutated, the mRNA does not splice out introns as normally would be the case. These introns can be translated into non-self peptides in patients having cancer.

### SUMMARY

The present disclosure provides pharmaceutical compositions comprising one or more peptides derived from ATaRIs.

The present disclosure also provides methods for treating a subject having cancer, wherein the subject comprises one or more deleterious mutations in the SETD2 gene, by administering one or more peptides derived from ATaRIs and/or one or more mRNA molecules encoding peptides derived from ATaRIs.

The present disclosure also provides methods of immunizing a subject against cancer or eliciting an immune response to one or more peptides derived from ATaRIs in a subject, wherein the subject comprises one or more deleterious mutations in the SETD2 gene, the method comprising administering to the subject one or more peptides derived from ATaRIs and/or one or more mRNA molecules encoding peptides derived from ATaRIs.

### DESCRIPTION OF EMBODIMENTS

By vaccinating patients against one or more peptides derived from ATaRIs expressed in tumor cells, the immune system may attack the tumor, since the tumor should be the main anatomic location where these introns will be expressed as proteins. Such a vaccine may be indicated for many or all patients with cancers that harbor deleterious SETD2 mutations. Such a vaccine may contain or encode peptide sequences or other forms of vaccine (such as mRNA or plasmid vaccines or dendritic cell vaccines) which are expected to be aberrantly translated as a result of intron retention. Ideally, the peptide sequences would be derived from introns that are retained/translated across a plurality or majority of human tumors. The vaccines can be administered in combination with other immunotherapies, such as immune checkpoint blockade, for the purpose of immunizing patients against their tumors.

It is possible that finding enough introns that are translated across a plurality of patients to make a cancer vaccine that is widely applicable (due to variations in HLA genotypes etc.) is not feasible. To overcome this problem, each patient's tumor can undergo sequencing such as, for example, by RNAseq to identify introns which may be translated to generate a vaccine. The vaccine can be tumor specific, thus reducing the likelihood of any autoimmune disease resulting from the vaccine.

In addition, it is proposed that the same vaccine cocktail (i.e., directed to a plurality of peptides) might be applicable to all patients with deleterious somatic mutations in SETD2. Previous neoantigen-based vaccine approaches typically require: a) whole exome sequencing to identify mutations that result from single nucleotide variations in combination with informatics analyses to predict neoantigens; and b) RNAseq to identify expressed neoantigens. For a majority of therapeutic neoantigen vaccines in clinical trials presently, these assays need to be performed on each subject enrolled in the trial. The present disclosure requires only one molecular feature to be characterized: SETD2 functional status (i.e. whether there is a deleterious mutation). This can be measured in panel sequencing assays, whole genome sequencing assays, whole exome sequencing assays, or potentially with immunohistochemistry of the tumor or RNA sequencing (e.g., intron profile of the tumor).

Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by ±10% and remain within the scope of the disclosed embodiments.

As used herein, the term “carrier” means a diluent, adjuvant, or excipient with which a compound is administered in a composition.

As used herein, the term, “compound” means all stereoisomers, tautomers, isotopes, and polymorphs of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the term “contacting” means bringing together two compounds, molecules, or entities in an in vitro system or an in vivo system.

As used herein, the terms “individual,” “subject,” and “patient,” used interchangeably, mean any animal described herein.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof. In some embodiments, the “individual,” “subject,” or “patient” is in an environment or will be traveling to an environment, or has traveled to an environment in which a particular disease, disorder, or condition is prevalent.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor, or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subject's response to treatment.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

As used herein, the terms “cancer” and “cancerous” refer to or describe the physiological condition in mammals that is typically characterized by unregulated cell growth/proliferation. A “tumor” comprises one or more cancerous cells. Examples of cancer are provided elsewhere herein.

As used herein, the terms “co-administration” and “co-administering” and “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

As used herein, the term “concurrently” means that a drug that is administered with one or more other drugs is administered during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The present disclosure provides pharmaceutical compositions comprising one or more peptides derived from ATaRIs. In some embodiments, the amino acid sequence of the ATaRI peptide comprises any one or more of SEQ ID NOs: 1-8675. In some embodiments, the pharmaceutical composition comprises a plurality of peptides derived from ATaRIs. In some embodiments, the pharmaceutical composition comprises at least two peptides having any of the amino acid sequences set forth in SEQ ID NOs: 1-8675. In some embodiments, the pharmaceutical composition comprises at least three peptides having any of the amino acid sequences set forth in SEQ ID NOs: 1-8675. In some embodiments, the pharmaceutical composition comprises at least four peptides having any of the amino acid sequences set forth in SEQ ID NOs: 1-8675. In some embodiments, the pharmaceutical composition comprises at least ten peptides having any of the amino acid sequences set forth in SEQ ID NOs: 1-8675. In some embodiments, the pharmaceutical composition comprises at least fifty peptides having any of the amino acid sequences set forth in SEQ ID NOs: 1-8675. In some embodiments, the pharmaceutical composition comprises at least one hundred peptides having any of the amino acid sequences set forth in SEQ ID NOs: 1-8675. In some embodiments, the pharmaceutical composition comprises at least five hundred peptides having any of the amino acid sequences set forth in SEQ ID NOs: 1-8675. In some embodiments, the pharmaceutical composition comprises at least one thousand peptides having any of the amino acid sequences set forth in SEQ ID NOs: 1-8675. In some embodiments, the pharmaceutical composition comprises at least two thousand peptides having any of the amino acid sequences set forth in SEQ ID NOs: 1-8675. In some embodiments, the pharmaceutical composition comprises at least three thousand peptides having any of the amino acid sequences set forth in SEQ ID NOs: 1-8675. In some embodiments, the pharmaceutical composition comprises at least four thousand peptides having any of the amino acid sequences set forth in SEQ ID NOs: 1-8675. In some embodiments, the pharmaceutical composition comprises at least five thousand peptides having any of the amino acid sequences set forth in SEQ ID NOs: 1-8675. In some embodiments, the pharmaceutical composition comprises at least six thousand peptides having any of the amino acid sequences set forth in SEQ ID NOs: 1-8675. In some embodiments, the pharmaceutical composition comprises at least seven thousand peptides having any of the amino acid sequences set forth in SEQ ID NOs: 1-8675. In some embodiments, the pharmaceutical composition comprises at least eight thousand peptides having any of the amino acid sequences set forth in SEQ ID NOs: 1-8675.

In some embodiments, any two or more of the peptides derived from ATaRIs described herein can be combined in the form of a fusion protein or encoded by one or more mRNA molecules or in one or more vectors. In some embodiments, any of the peptides derived from ATaRIs described herein, or fusion proteins comprising the same, can have an amino acid sequence that is 100%, or from 70% to 99.9%, identical to the particular amino acid sequences set forth in SEQ ID NOs: 1-8675. The amino acid sequence of any individual peptide, or fusion proteins comprising the same, can be at least 70%, at least 75%, at least 80%, at least 85%, at least 90%, at least 95%, at least 96%, at least 97%, at least 98%, or at least 99% identical to the particular amino acid sequences set forth in SEQ ID NOs: 1-8675. Identity or similarity with respect to an amino acid or nucleotide sequence is defined herein as the percentage of amino acid residues (or nucleotide residues as the case may be) in the particular peptide or fusion protein that are identical (i.e., same residue) with the amino acid or nucleotide sequence for the peptide or fusion protein having particular amino acid sequences set forth in SEQ ID NOs: 1-8675, after aligning the sequences and introducing gaps, if necessary, to achieve the maximum percent sequence identity. Percent sequence identity can be determined by, for example, the Gap program (Wisconsin Sequence Analysis Package, Version 8 for Unix, Genetics Computer Group, University Research Park, Madison WI), using default settings, which uses the algorithm of Smith and Waterman (Adv. Appl. Math., 1981, 2, 482-489). Any amino acid number calculated as a % identity can be rounded up or down, as the case may be, to the closest whole number.

Any of the peptides derived from ATaRIs described herein, or fusion proteins comprising the same, can be fragments of the particular amino acid sequences set forth in SEQ ID NOs: 1-8675. The amino acid sequence of any of the peptides derived from ATaRIs described herein, or fusion proteins comprising the same, can be missing consecutive amino acids constituting at least 20%, at least 15%, at least 10%, at least 5%, at least 4%, at least 3%, at least 2%, or at least 1%, of the particular amino acid sequences set forth in SEQ ID NOs: 1-8675. The omitted consecutive amino acids may be from the C-terminus or N-terminus portion of the peptide. Alternately, the omitted consecutive amino acids may be from the internal portion of the peptide, thus retaining at least its C-terminus and N-terminus amino acids of the peptide. In some embodiments, the fragments may comprise 10 amino acids, 20 amino acids, 30 amino acids, 40 amino acids, or 50 amino acids of any of the particular amino acid sequences set forth in SEQ ID NOs: 1-8675.

Any of the peptides derived from ATaRIs described herein, or fusion proteins comprising the same, can have one or more amino acid additions, deletions, or substitutions compared to the particular amino acid sequences set forth in SEQ ID NOs: 1-8675. Any of the peptides derived from ATaRIs described herein, or fusion proteins comprising the same, can have at least one, at least two, at least three, at least four, at least five, at least six, at least seven, at least eight, at least nine, at least ten, at least eleven, or at least twelve amino acid additions, deletions, or substitutions compared to the particular amino acid sequences set forth in SEQ ID NOs: 1-8675. In some embodiments, the peptides derived from ATaRIs described herein, or fusion proteins comprising the same, have at least one, at least two, at least three, at least four, at least five, or at least six amino acid additions, deletions, or substitutions compared to the particular amino acid sequences set forth in SEQ ID NOs: 1-8675. Any of the peptides derived from ATaRIs described herein, or fusion proteins comprising the same, can have one, two, three, four, five, six, seven, eight, nine, ten, eleven, or twelve amino acid additions, deletions, or substitutions compared to the particular amino acid sequences set forth in SEQ ID NOs: 1-8675. In some embodiments, the peptides derived from ATaRIs described herein, or fusion proteins comprising the same, have one, two, three, four, five, or six amino acid additions, deletions, or substitutions compared to the particular amino acid sequences set forth in SEQ ID NOs: 1-8675. The amino acid additions, deletions, or substitutions can take place at any amino acid position within the peptide derived from ATaRIs described herein.

Where a particular peptide derived from ATaRIs described herein, or fusion protein comprising the same, comprises at least one or more substitutions, the substituted amino acid(s) can each be, independently, any naturally occurring amino acid or any non-naturally occurring amino acid. Thus, a particular peptide derived from ATaRIs described herein may comprise one or more amino acid substitutions that are naturally occurring amino acids and/or one or more amino acid substitutions that are non-naturally occurring amino acids. Individual amino acid substitutions are selected from any one of the following: 1) the set of amino acids with nonpolar sidechains, for example, Ala, Cys, Ile, Leu, Met, Phe, Pro, Val; 2) the set of amino acids with negatively charged side chains, for example, Asp, Glu; 3) the set of amino acids with positively charged sidechains, for example, Arg, His, Lys; and 4) the set of amino acids with uncharged polar sidechains, for example, Asn, Cys, Gln, Gly, His, Met, Phe, Ser, Thr, Trp, Tyr, to which are added Cys, Gly, Met and Phe. Substitutions of a member of one class with another member of the same class are contemplated herein. Naturally occurring amino acids include, for example, alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Non-naturally occurring amino acids include, for example, norleucine, ornithine, norvaline, homoserine, and other amino acid residue analogues such as those described in Ellman et al., Meth. Enzym., 1991, 202, 301-336. To generate such non-naturally occurring amino acid residues, the procedures of Noren et al., Science, 1989, 244, 182 and Ellman et al., supra, can be used. Briefly, these procedures involve chemically activating a suppressor tRNA with a non-naturally occurring amino acid residue followed by in vitro transcription and translation of the RNA.

The present disclosure also provides nucleic acid molecules encoding any of the peptides derived from ATaRIs described herein, or fusion proteins comprising the same. One skilled in the art having knowledge of the genetic code can routinely prepare and design a plethora of nucleic acid molecules encoding the same peptide derived from ATaRIs described herein, or fusion protein comprising the same. The length and nucleotide content of any particular nucleic acid molecule is dictated by the desired amino acid sequence of the encoded peptide derived from ATaRIs described herein, or fusion protein comprising the same. The nucleic acid molecules can comprise DNA and/or RNA. In some embodiments, the nucleic acid molecules can comprise mRNA molecules encoding any of the peptides derived from ATaRIs described herein, or fusion proteins comprising the same. Such mRNA molecules can be used as vaccines.

The present disclosure also provides vectors encoding any of the peptides derived from ATaRIs described herein, or fusion proteins comprising the same. The vector can be capable of expressing any of the peptides derived from ATaRIs described herein, or fusion proteins comprising the same, in the cell of a mammal in a quantity effective to elicit an immune response in the mammal. The vector can be recombinant. The vector can be a plasmid. In some embodiments, the plasmid is a DNA plasmid. The vector can be useful for transfecting cells with nucleic acid encoding any of the peptides derived from ATaRIs described herein, or fusion proteins comprising the same, which the transformed host cell is cultured and maintained under conditions wherein expression of the peptide or fusion protein takes place. In some embodiments, mRNA or peptides can be loaded into a dendritic cell vaccine.

In some embodiments, the vector is a non-viral vector. In some embodiments, the non-viral vector is RNA, such as mRNA. In some embodiments, the mRNA is protamine-complexed mRNA, wherein the peptide or fusion protein is encoded by the mRNA, and the protamine complexes contribute a strong immunostimulatory signal. An exemplary mRNA vector platform is RNActive® (CureVac Inc).

In some embodiments, coding sequences can be optimized for stability and high levels of expression. In some instances, codons are selected to reduce secondary structure formation of the RNA such as that formed due to intramolecular bonding.

In some embodiments, the vectors can comprise regulatory elements for gene expression of the coding sequences of the nucleic acid. The regulatory elements can be a promoter, an enhancer an initiation codon, a stop codon, a polyadenylation signal, or elements that drive high expression of encoded molecules. In some embodiments, the vector can comprise heterologous nucleic acid encoding any of the peptides derived from ATaRIs described herein, or fusion proteins comprising the same, and can further comprise an initiation codon, which is upstream of the peptide coding sequence, and a stop codon, which is downstream of the peptide coding sequence. The initiation and termination codon are in frame with the peptide coding sequence.

The vector can also comprise a promoter that is operably linked to the peptide or fusion protein coding sequence. The promoter operably linked to the peptide or fusion protein coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter, or the like. The promoter can also be a promoter from a human gene such as human actin, human myosin, human hemoglobin, human muscle creatine, or human metallothionein. The promoter can also be a tissue specific promoter, such as a muscle or skin specific promoter, natural or synthetic. Representative examples of promoters include the bacteriophage T7 promoter, bacteriophage T3 promoter, SP6 promoter, lac operator-promoter, tac promoter, mycobacterial Hsp60 promoter, SV40 late promoter, SV40 early promoter, RSV-LTR promoter, CMV IE promoter, SV40 early promoter or SV40 late promoter and the CMV IE promoter.

The vector can also comprise a polyadenylation signal, which can be downstream of the peptide or fusion protein coding sequence. The polyadenylation signal can be a SV40 polyadenylation signal, LTR polyadenylation signal, CMV polyadenylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, CA).

The vector can also comprise an enhancer upstream of the peptide encoding sequences. The enhancer can be necessary for DNA expression. The enhancer can be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737. The vector can also comprise a mammalian origin of replication in order to maintain the vector extrachromosomally and produce multiple copies of the vector in a cell.

The vector can also comprise a regulatory sequence, which can be well suited for gene expression in a mammalian or human cell into which the vector is administered. The consensus coding sequence can comprise a codon, which can allow more efficient transcription of the coding sequence in the host cell.

The vector can be pSE420 (Invitrogen, San Diego, Calif.) or pET28b (EMD Millipore, Billerca, Mass.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which may be used for protein production in mammalian cells such as Chinese hamster ovary (CHO) cells. The vector can be expression vectors or systems to produce protein by routine techniques and readily available starting materials including Sambrook et al., Molecular Cloning and Laboratory Manual, Second Ed., Cold Spring Harbor (1989).

In some embodiments, the vector is a viral vector. Suitable viral vectors include, but are not limited to, an adenovirus vector, an adeno-associated virus vector, a poxvirus vector (such as, for example, vaccinia virus vector), a paramyxovirus vector, a fowlpox virus vector, an attenuated yellow fever vectors (such as, for example, YFV-17D), an alphavirus vector, a retrovirus vector (such as, for example, lentivirus vector), a Sendai virus vector, and cytomegalovirus (CMV) vector. Suitable adenovirus vectors include, but are not limited to, adenovirus 4, adenovirus 5, chimpanzee adenovirus 3, chimpanzee adenovirus 63, and chimpanzee adenovirus 68. A suitable vaccinia virus vector includes, but is not limited to, modified vaccinia Ankara (MVA). Suitable paramyxovirus vectors include, but are not limited to, modified parainfluenza virus (PIV2) and recombinant human parainfluenza virus (rHPIV2). Suitable CMV vectors include, but are not limited to, Rhesus Macaque CMV (RhCMV) vectors and Human CMV (HCMV) vectors. In some embodiments, the vector is present within a composition comprising a pharmaceutically acceptable carrier. One skilled in the art is readily familiar with numerous vectors, many of which are commercially available.

The present disclosure also provides host cells comprising any of the nucleic acid molecules or vectors disclosed herein. The host cells can be used, for example, to express the peptides or fusion proteins, or fragments of thereof. The peptides of fusion proteins, or fragments thereof, can also be expressed in cells in vivo. The host cell that is transformed (for example, transfected) to produce the peptides or fusion proteins, or fragments of thereof, can be an immortalized mammalian cell line, such as those of lymphoid origin (for example, a myeloma, hybridoma, trioma or quadroma cell line). The host cell can also include normal lymphoid cells, such as B-cells, that have been immortalized by transformation with a virus (for example, the Epstein-Barr virus).

In some embodiments, the host cells include, but are not limited to: bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda (for example, Sf9 and Sf21 cell lines, and expresSF™ cells (Protein Sciences Corp., Meriden, CT, USA)), Drosophila S2 cells, and Trichoplusia in High Five® Cells (Invitrogen, Carlsbad, CA, USA); and mammalian cells, such as COS1 and COS7 cells, Chinese hamster ovary (CHO) cells, NSO myeloma cells, NIH 3T3 cells, 293 cells, Procell92S, perC6, HEPG2 cells, HeLa cells, L cells, HeLa, MDCK, HEK293, WI38, murine ES cell lines (for example, from strains 129/SV, C57/BL6, DBA-1, 129/SVJ), K562, Jurkat cells, and BW5147. Other useful mammalian cell lines are well known and readily available from the American Type Culture Collection (“ATCC”) (Manassas, VA, USA) and the National Institute of General Medical Sciences (NIGMS) Human Genetic Cell Repository at the Coriell Cell Repositories (Camden, NJ, USA). These cell types are only representative and are not meant to be an exhaustive list.

Among other considerations, some of which are described above, a host cell strain may be chosen for its ability to process the expressed peptide or fusion protein, or fragment thereof, in the desired fashion. Post-translational modifications of the polypeptide include, but are not limited to, glycosylation, acetylation, carboxylation, phosphorylation, lipidation, and acylation.

In some embodiments, the cell comprising the one or more vector(s) is present within a composition comprising a pharmaceutically acceptable carrier.

The present disclosure also provides any of the peptides derived from ATaRIs described herein, or fusion proteins comprising the same, in which the composition comprises at least one nucleic acid molecule encoding at least one of the peptides or fusion proteins. In some embodiments, the composition comprises one peptide or fusion protein in protein form and one or two nucleic acid molecules encoding two peptides or fusion proteins. In some embodiments, the composition comprises two peptides or fusion proteins in protein form, and one nucleic acid molecule encoding one peptide or fusion protein. Thus, the present composition can be a mixture of a protein form of any of the peptides derived from ATaRIs described herein, or fusion proteins comprising the same, and nucleic acid molecule(s) encoding any of the peptides derived from ATaRIs described herein, or fusion proteins comprising the same.

The present disclosure also provides compositions comprising any one or more of the peptides, fusion proteins, or nucleic acid molecules encoding the same, cells, and/or vectors and a pharmaceutically acceptable carrier. Compositions include, for example, pharmaceutical compositions. A pharmaceutically acceptable carrier refers to at least one component of a pharmaceutical preparation that is normally used for administration of active ingredients. As such, a carrier can contain any pharmaceutical excipient used in the art and any form of vehicle for administration. Carriers include, but are not limited to, phosphate buffered saline, physiological saline, water, citrate/sucrose/Tween formulations and emulsions such as, for example, oil/water emulsions.

In some embodiments, the pharmaceutical composition further comprises one or more immune checkpoint inhibitors or one or more chemotherapeutic agents, or any combination thereof. In some embodiments, the pharmaceutical composition further comprises one or more immune checkpoint inhibitors. In some embodiments, the pharmaceutical composition further comprises one or more chemotherapeutic agents.

In some embodiments, the pharmaceutical composition further comprises one or more immune checkpoint inhibitors and one or more chemotherapeutic agents.

In some embodiments, the immune checkpoint inhibitor comprises a PD-1 checkpoint inhibitor, a PD-L1 checkpoint inhibitor, a CTLA-4 checkpoint inhibitor, a TIGIT checkpoint inhibitor, or a LAG-3 checkpoint inhibitor, or any combination thereof. In some embodiments, the immune checkpoint inhibitor comprises a PD-1 checkpoint inhibitor. In some embodiments, the immune checkpoint inhibitor comprises a PD-L1 checkpoint inhibitor. In some embodiments, the immune checkpoint inhibitor comprises a CTLA-4 checkpoint inhibitor. In some embodiments, the immune checkpoint inhibitor comprises a TIGIT checkpoint inhibitor. In some embodiments, the immune checkpoint inhibitor comprises a LAG-3 checkpoint inhibitor.

In some embodiments, the PD-1 checkpoint inhibitor comprises nivolumab, pembrolizumab, cetrelimab, or cemiplimab, or any combination thereof. In some embodiments, the PD-1 checkpoint inhibitor comprises nivolumab. In some embodiments, the PD-1 checkpoint inhibitor comprises pembrolizumab. In some embodiments, the PD-1 checkpoint inhibitor comprises cetrelimab. In some embodiments, the PD-1 checkpoint inhibitor comprises cemiplimab.

In some embodiments, the PD-L1 checkpoint inhibitor comprises atezolizumab, durvalab, or avelumab, or any combination thereof. In some embodiments, the PD-L1 checkpoint inhibitor comprises atezolizumab. In some embodiments, the PD-L1 checkpoint inhibitor comprises durvalab. In some embodiments, the PD-L1 checkpoint inhibitor comprises avelumab

In some embodiments, the CTLA-4 checkpoint inhibitor comprises ipilumumab or tremelimumab, or a combination thereof. In some embodiments, the CTLA-4 checkpoint inhibitor comprises ipilumumab. In some embodiments, the CTLA-4 checkpoint inhibitor comprises tremelimumab.

In some embodiments, the chemotherapeutic agent comprises a tyrosine kinase inhibitor (TKI). In some embodiments, the TKI comprises bevacizumab, sunitinib, sorafenib, pazopanib, cabozantinib, lenvatinib, axitinib, or tivozanib, or any combination thereof. In some embodiments, the TKI comprises bevacizumab. In some embodiments, the TKI comprises sunitinib. In some embodiments, the TKI comprises sorafenib. In some embodiments, the TKI comprises pazopanib. In some embodiments, the TKI comprises cabozantinib. In some embodiments, the TKI comprises Lenvatinib. In some embodiments, the TKI comprises axitinib. In some embodiments, the TKI comprises tivozanib.

In some embodiments, the chemotherapeutic agent comprises a mammalian target of rapamycin (mTOR) inhibitor. In some embodiments, the mTOR inhibitor comprises temsirolimus or everolimus, or a combination thereof. In some embodiments, the mTOR inhibitor comprises temsirolimus. In some embodiments, the mTOR inhibitor comprises everolimus.

In some embodiments, the pharmaceutical composition is an oral dosage form, an intravenous dosage form, a topical dosage form, an intraperitoneal dosage form, or an intrathecal dosage form. In some embodiments, the pharmaceutical composition is an oral dosage form or an intravenous dosage form. In some embodiments, the pharmaceutical composition is an oral dosage form.

In some embodiments, the oral dosage form is a pill, tablet, capsule, cachet, gel-cap, pellet, powder, granule, or liquid. In some embodiments, the oral dosage form is a pill, tablet, capsule, gel-cap, or liquid. In some embodiments, the oral dosage form is a pill. In some embodiments, the oral dosage form is a tablet. In some embodiments, the oral dosage form is a capsule. In some embodiments, the oral dosage form is a gel-cap. In some embodiments, the oral dosage form is a liquid.

In some embodiments, the oral dosage form is protected from light and present within a blister pack, bottle, or intravenous bag. In some embodiments, the oral dosage form is present within a blister pack, bottle, or intravenous bag. In some embodiments, the oral dosage form is present within a blister pack. In some embodiments, the oral dosage form is present within a bottle. In some embodiments, the oral dosage form is present within an intravenous bag.

The compounds and compositions of the therapeutic vaccines described herein can be formulated for parenteral administration by injection, such as by bolus injection or continuous infusion. The compounds and compositions can be administered by continuous infusion subcutaneously over a period of about 15 minutes to about 24 hours. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions of the therapeutic vaccines described herein can be formulated by combining the compounds with pharmaceutically acceptable carriers. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a subject to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compounds and compositions of the therapeutic vaccines can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

In transdermal administration, the compounds and compositions of the therapeutic vaccines can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The compounds and compositions of the therapeutic vaccines described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds and compositions of the therapeutic vaccines can be delivered in a controlled release system. In some embodiments, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

The compounds and compositions of the therapeutic vaccines described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

Suitable compositions include, but are not limited to, oral non-absorbed compositions. Suitable compositions also include, but are not limited to saline, water, cyclodextrin solutions, and buffered solutions of pH 3-9.

The compounds and compositions of the therapeutic vaccines described herein can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris (hydroxymethyl) amino methane HCl (pH7.0), 0.9% saline, and 1.2% saline, and any combination thereof. In some embodiments, excipient is chosen from propylene glycol, purified water, and glycerin.

In some embodiments, the compounds and compositions of the therapeutic vaccines described herein can be lyophilized to a solid and reconstituted with, for example, water prior to use.

When administered to a human, the compounds and compositions of the therapeutic vaccines can be sterile. Water is a suitable carrier when the compound and composition is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions of the therapeutic vaccines described herein can take the form of a solution, suspension, emulsion, tablet, pill, pellet, capsule, capsule containing a liquid, powder, sustained-release formulation, aerosol, spray, or any other form suitable for use. Examples of suitable pharmaceutical carriers are described in Remington's Pharmaceutical Sciences, A.R. Gennaro (Editor) Mack Publishing Co.

In some embodiments, the compounds and compositions of the therapeutic vaccines are formulated in accordance with routine procedures as pharmaceutical compositions adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound or composition is to be administered by infusion, it can be dispensed, for example, with an infusion bottle containing sterile pharmaceutical grade water or saline. Where the compound or composition is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition is in the form of a liquid wherein the active agents are present in solution, in suspension, as an emulsion, or as a solution/suspension. In some embodiments, the liquid composition is in the form of a gel. In other embodiments, the liquid composition is aqueous. In other embodiments, the composition is in the form of an ointment.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone (Povidone™), hydroxypropyl methylcellulose, sucrose, starch, and ethylcellulose. Lubricants that can be used include magnesium stearates, stearic acid, silicone fluid, talc, waxes, oils, and colloidal silica. Additional excipients include, for example, colorants, taste-masking agents, solubility aids, suspension agents, compressing agents, enteric coatings, sustained release aids, and the like.

In some embodiments, the compositions of the therapeutic vaccines can be administered in the form of a depot injection or implant preparation, which can be formulated in such a manner as to permit a sustained release. An exemplary composition comprises any one or more of the compositions described herein formulated in aqueous buffer.

In some embodiments, liquid formulations of a pharmaceutical composition for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. Liquid formulations of pharmaceutical compositions can also include solutions, emulsions, syrups and elixirs containing, together with the active compound(s), wetting agents, sweeteners, and coloring and flavoring agents. Various liquid and powder formulations of the pharmaceutical compositions can be prepared by conventional methods for inhalation into the lungs of the mammal to be treated.

In some embodiments, liquid formulations of a pharmaceutical composition for injection can comprise various carriers such as vegetable oils, dimethylacetamide, dimethylformamide, ethyl lactate, ethyl carbonate, isopropyl myristate, ethanol, polyols such as, for example, glycerol, propylene glycol, liquid polyethylene glycol, and the like. In some embodiments, the composition includes a citrate/sucrose/tween carrier. For intravenous injections, water soluble versions of the compositions can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution or other suitable excipients. A suitable insoluble form of the composition can be prepared and administered as a suspension in an aqueous base or a pharmaceutically acceptable oil base, such as an ester of a long chain fatty acid such as, for example, ethyl oleate.

The compositions of the therapeutic vaccines can be, for example, injectable solutions, aqueous suspensions or solutions, non-aqueous suspensions or solutions, solid and liquid oral formulations, salves, gels, ointments, intradermal patches, creams, aerosols, lotions, tablets, capsules, sustained release formulations, and the like. In some embodiments, for topical applications, the pharmaceutical compositions can be formulated in a suitable ointment. In some embodiments, a topical semi-solid ointment formulation typically comprises a concentration of the active ingredient from about 1 to 20%, or from 5 to 10%, in a carrier, such as a pharmaceutical cream base. Some examples of formulations of a composition for topical use include, but are not limited to, drops, tinctures, lotions, creams, solutions, and ointments containing the active ingredient and various supports and vehicles.

In some embodiments, any of the peptides or fusion proteins, or vectors encoding the same, or cells described herein, or compositions comprising the same, can be administered to a mammal as an aerosol. In some embodiments, the aerosol inocula comprises saline. Conventional aerosol delivery devices include, but are not limited to, a pressurized metered dose inhaler (pMDI) and a dry power inhaler (DPI), both of which deliver a dry powder formulation, and nebulizers such as the PARI eFlow device, which delivers an aqueous dose as a fine mist. In some embodiments, the aerosol delivery device is a Pari eFlow portable electronic aerosol delivery platform attached to a delivery mask. In some embodiments, the average particle size is from about 1 μm to about 10 μm, from about 1 μm to about 5 μm, from about 3 μm to about 5 μm, from about 4 μm to about 5 μm, or from about 3.9 μm to about 4.9 μm. In some embodiments, the aerosol is in a volume from about 0.1 ml to about 5 ml, from about 0.1 ml to about 2 ml, from about 0.1 ml to about 1.5 ml, from about 0.5 ml to about 1.5 ml, from about 0.5 ml to about 1.2 ml, from about 0.7 ml to about 1.2 ml, or about 1 ml.

The compositions of the therapeutic vaccines can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freund's incomplete adjuvant, LPS analog including monophosphoryl lipid A, muramyl peptides, quinone analogs, vesicles such as squalene and squalane, hyaluronic acid, lipids, liposomes, calcium ions, viral proteins, polyanions, polycations, or nanoparticles, or other known transfection facilitating agents. The transfection facilitating agent is a polyanion, polycation, including poly-L-glutamate (LGS), or lipid. The transfection facilitating agent is poly-L-glutamate, and more suitably, the poly-L-glutamate is present in the composition at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalane, and hyaluronic acid can also be used administered in conjunction with the genetic construct. In some embodiments, the plasmid compositions can also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be an adjuvant. The adjuvant may be other genes that are expressed in alternative plasmid or are delivered as proteins in combination with the plasmid above. The adjuvant may be selected from the group consisting of: a-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. The adjuvant may be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes which may be useful adjuvants include those encoding: MCP-1, MIP-la, MIP-1p, IL-8, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The present disclosure also provides kits comprising any of the peptides or fusion proteins, nucleic acid molecules, vectors, or cells, described herein. The kit can include, for example, container(s), package(s) or dispenser(s) along with labels and instructions for administration or use.

The present disclosure also provides methods of treating a subject having cancer, wherein the subject comprises one or more deleterious mutations in the SETD2 gene, the methods comprising administering to the subject one or more peptides derived from ATaRIs and/or one or more mRNA molecules encoding peptides derived from ATaRIs.

The present disclosure also provides methods of immunizing a subject against cancer or eliciting an immune response to one or more peptides derived from ATaRIs in a subject, wherein the subject comprises one or more deleterious mutations in the SETD2 gene, the methods comprising administering to the subject one or more peptides derived from ATaRIs and/or one or more mRNA molecules encoding peptides derived from ATaRIs.

In some embodiments, the methods further comprising analyzing a biological sample obtained from the subject for the presence of the one or more deleterious mutations in the SETD2 gene.

In some embodiments, the amino acid sequence of the one or more peptides derived from ATaRI are selected from the group consisting of SEQ ID NOs: 1-8675. In some embodiments, two or more peptides are in the form of a fusion protein as described herein.

In some embodiments, the cancer is SETD2-mutant liver cancer, mesothelioma, lung cancer, or kidney cancer. In some embodiments, the cancer is SETD2-mutant liver cancer. In some embodiments, the cancer is SETD2-mutant mesothelioma. In some embodiments, the cancer is SETD2-mutant lung cancer. In some embodiments, the cancer is SETD2-mutant kidney cancer. In some embodiments, the kidney cancer is clear cell renal cell carcinoma or papillary renal cell carcinoma. In some embodiments, the kidney cancer is clear cell renal cell carcinoma. In some embodiments, the kidney cancer is papillary renal cell carcinoma.

In some embodiments, the biological sample is a tumor sample.

In some embodiments, the presence of the one or more deleterious mutations in the SETD2 gene is detected by nucleic acid sequencing. In some embodiments, the nucleic acid sequencing is RNA sequencing.

In some embodiments, the methods further comprise administering to the subject one or more immune checkpoint inhibitors or one or more chemotherapeutic agents, or any combination thereof. In some embodiments, the one or more immune checkpoint inhibitors or one or more chemotherapeutic agents, or any combination thereof, may be present in the same pharmaceutical composition containing the peptide(s) or fusion protein(s), or vectors encoding the same. In some embodiments, the one or more immune checkpoint inhibitors or one or more chemotherapeutic agents, or any combination thereof, may be present in separate pharmaceutical compositions than the pharmaceutical compositions containing the peptide(s) or fusion protein(s), or vectors encoding the same.

In some embodiments, the immune checkpoint inhibitor comprises a PD-1 checkpoint inhibitor, a PD-L1 checkpoint inhibitor, a CTLA-4 checkpoint inhibitor, a TIGIT checkpoint inhibitor, or a LAG-3 checkpoint inhibitor, or any combination thereof. In some embodiments, the immune checkpoint inhibitor comprises a PD-1 checkpoint inhibitor. In some embodiments, the immune checkpoint inhibitor comprises a PD-L1 checkpoint inhibitor. In some embodiments, the immune checkpoint inhibitor comprises a CTLA-4 checkpoint inhibitor. In some embodiments, the immune checkpoint inhibitor comprises a TIGIT checkpoint inhibitor. In some embodiments, the immune checkpoint inhibitor comprises a LAG-3 checkpoint inhibitor.

In some embodiments, the PD-1 checkpoint inhibitor comprises nivolumab, pembrolizumab, cetrelimab, or cemiplimab, or any combination thereof. In some embodiments, the PD-1 checkpoint inhibitor comprises nivolumab. In some embodiments, the PD-1 checkpoint inhibitor comprises pembrolizumab. In some embodiments, the PD-1 checkpoint inhibitor comprises cetrelimab. In some embodiments, the PD-1 checkpoint inhibitor comprises cemiplimab.

In some embodiments, the PD-L1 checkpoint inhibitor comprises atezolizumab, durvalab, or avelumab, or any combination thereof. In some embodiments, the PD-L1 checkpoint inhibitor comprises atezolizumab. In some embodiments, the PD-L1 checkpoint inhibitor comprises durvalab. In some embodiments, the PD-L1 checkpoint inhibitor comprises avelumab.

In some embodiments, the CTLA-4 checkpoint inhibitor comprises ipilumumab or tremelimumab, or a combination thereof. In some embodiments, the CTLA-4 checkpoint inhibitor comprises ipilumumab. In some embodiments, the CTLA-4 checkpoint inhibitor comprises tremelimumab.

In some embodiments, the chemotherapeutic agent comprises a tyrosine kinase inhibitor (TKI). In some embodiments, the TKI comprises bevacizumab, sunitinib, sorafenib, pazopanib, cabozantinib, lenvatinib, axitinib, or tivozanib, or any combination thereof. In some embodiments, the TKI comprises bevacizumab. In some embodiments, the TKI comprises sunitinib. In some embodiments, the TKI comprises sorafenib. In some embodiments, the TKI comprises pazopanib. In some embodiments, the TKI comprises cabozantinib. In some embodiments, the TKI comprises lenvatinib. In some embodiments, the TKI comprises axitinib. In some embodiments, the TKI comprises tivozanib.

In some embodiments, the chemotherapeutic agent comprises a mammalian target of rapamycin (mTOR) inhibitor. In some embodiments, the mTOR inhibitor comprises temsirolimus or everolimus, or a combination thereof. In some embodiments, the mTOR inhibitor comprises temsirolimus. In some embodiments, the mTOR inhibitor comprises everolimus.

In some embodiments, the methods further comprise administering another immunotherapy to the subject. In some embodiments, the another immunotherapy comprises chimeric antigen receptor-T cells (CAR-T), bone marrow transplant, adoptive transfer, interleukin-2, or interferon, or any combination thereof. In some embodiments, the another immunotherapy comprises chimeric antigen receptor-T cells (CAR-T). In some embodiments, the another immunotherapy comprises bone marrow transplant. In some embodiments, the another immunotherapy comprises adoptive transfer. In some embodiments, the another immunotherapy comprises interleukin-2.

The compounds and compositions described herein can be administered by any route of administration including, but not limited to, oral, intravenous, topical, intraperitoneal, and intrathecal. In some embodiments, the administration is oral, intravenous, intraperitoneal, or intrathecal. In some embodiments, the administration is oral, intravenous, or intraperitoneal. In some embodiments, the administration is oral or intravenous. In some embodiments, the administration is oral or topical. In some embodiments, the administration is oral or intraperitoneal. In some embodiments, the administration is oral or intrathecal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, or composition(s) comprising the same to a particular area in need of treatment. This may be achieved, for example, by local infusion (for example, during surgery), topical application (for example, with a wound dressing after surgery), or by injection (for example, by depot injection). Formulations for injection can be presented in unit dosage form, such as in ampoules or in multi-dose containers, with an added preservative.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The amount of any particular compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

Effective doses of the compositions of the present disclosure, for the treatment of a condition vary depending upon many different factors, including means of administration, target site, physiological state of the subject, whether the subject is human or an animal, other medications administered, and whether treatment is prophylactic or therapeutic. Usually, the subject is a human but non-human mammals including transgenic mammals can also be treated.

In some embodiments, the compositions can be administered to a subject by injection intravenously, subcutaneously, intraperitoneally, intramuscularly, intramedullarily, intraventricularly, intraepidurally, intraarterially, intravascularly, intraarticularly, intrasynovially, intrasternally, intrathecally, intrahepatically, intraspinally, intratumorly, intracranially, enteral, intrapulmonary, transmucosal, intrauterine, sublingual, or locally at sites of inflammation or tumor growth by using standard methods. In some embodiments, the compositions can be administered to a subject by injection intravenously. Alternately, the compositions can be administered to a subject by routes including oral, nasal, ophthalmic, rectal, or topical. The most typical route of administration is intravascular, subcutaneous, or intramuscular, although other routes can be effective. In some embodiments, compositions are administered as a sustained release composition or device, such as a Medipad™ device. The composition can also be administered via the respiratory tract, for example, using a dry powder inhalation device, nebulizer, or a metered dose inhaler. The composition can also be administered by traditional syringes, needleless injection devices, “microprojectile bombardment gone guns,” or other physical methods such as electroporation (“EP”), “hydrodynamic method”, or ultrasound.

In some embodiments, the composition can be administered to a subject by sustained release administration, by such means as depot injections of erodible implants directly applied during surgery or by implantation of an infusion pump or a biocompatible sustained release implant into the subject. Alternately, the composition can be administered to a subject by injectable depot routes of administration, such as by using 1-, 3-, or 6-month depot injectable or biodegradable materials and methods, or by applying to the skin of the subject a transdermal patch containing the composition, and leaving the patch in contact with the subject's skin, generally for 1 to 5 hours per patch.

In some embodiments, the compositions comprise about 1 nanogram to about 10 mg of nucleic acid molecule or peptide or fusion protein. In some embodiments, the compositions comprise: 1) at least 10, 15, 20, 25, 30, 35, 40, 45, 50, 55, 60, 65, 70, 75, 80, 85, 90, 95 or 100 nanograms, or at least 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms, or at least 1.5, 2, 2.5, 3, 3.5, 4, 4.5, 5, 5.5, 6, 6.5, 7, 7.5, 8, 8.5, 9, 9.5 or 10 mg or more; and 2) up to and including 15, 20, 25, 30, 35, 40, 45, 50, 55, 60, 65, 70, 75, 80, 85, 90, 95 or 100 nanograms, or up to and including 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms, or up to and including 1.5, 2, 2.5, 3, 3.5, 4, 4.5, 5, 5.5, 6, 6.5, 7, 7.5, 8, 8.5, 9, 9.5 or 10 mg.

In some embodiments, the compositions comprise about 5 ng to about 10 mg of nucleic acid molecule or peptide or fusion protein. In some embodiments, the compositions comprise about 25 ng to about 5 mg of nucleic acid molecule or peptide or fusion protein. In some embodiments, the compositions contain about 50 ng to about 1 mg of nucleic acid molecule or peptide or fusion protein. In some embodiments, the compositions contain about 0.1 to about 500 μg of nucleic acid molecule or peptide or fusion protein. In some embodiments, the compositions contain about 1 μg to about 350 μg of nucleic acid molecule or peptide or fusion protein. In some embodiments, the compositions contain about 5 μg to about 250 μg of nucleic acid molecule or peptide or fusion protein. In some embodiments, the compositions contain about 10 μg to about 200 μg of nucleic acid molecule or peptide or fusion protein. In some embodiments, the compositions contain about 15 μg to about 150 μg of nucleic acid molecule or peptide or fusion protein. In some embodiments, the compositions contain about 20 μg to about 100 μg of nucleic acid molecule or peptide or fusion protein. In some embodiments, the compositions contain about 25 μg to about 75 μg of nucleic acid molecule or peptide or fusion protein. In some embodiments, the compositions contain about 30 μg to about 50 μg of nucleic acid molecule or peptide or fusion protein. In some embodiments, the compositions contain about 35 μg to about 40 μg of nucleic acid molecule or peptide or fusion protein. In some embodiments, the compositions contain about 100 μg to about 200 μg of nucleic acid molecule or peptide or fusion protein. In some embodiments, the compositions comprise about 10 μg to about 100 μg of nucleic acid molecule or peptide or fusion protein. In some embodiments, the compositions comprise about 20 μg to about 80 μg of nucleic acid molecule or peptide or fusion protein. In some embodiments, the compositions comprise about 25 μg to about 60 μg of nucleic acid molecule or peptide or fusion protein. In some embodiments, the compositions comprise about 30 ng to about 50 μg of nucleic acid molecule or peptide or fusion protein. In some embodiments, the compositions comprise about 35 ng to about 45 μg of nucleic acid molecule or peptide or fusion protein. In some embodiments, the compositions contain about 0.1 μg to about 500 μg of nucleic acid molecule or peptide or fusion protein. In some embodiments, the compositions contain about 1 μg to about 350 μg of nucleic acid molecule or peptide or fusion protein. In some embodiments, the compositions contain about 25 μg to about 250 μg of nucleic acid molecule or peptide or fusion protein. In some embodiments, the compositions contain about 100 μg to about 200 μg of nucleic acid molecule or peptide or fusion protein.

In some embodiments, the delivery platforms described herein can be used either in a single administration alone or in combinations as matched peptide/fusion protein prime-boost approaches. For example, the same peptide/fusion protein can be used as both the prime and the boost. In other embodiments, a first peptide/fusion protein can be used as the prime and a second different peptide/fusion protein can be used as the boost (i.e., heterologous prime-boost). In some embodiments, the prime is a DNA or RNA (such as mRNA) prime and the boost is a viral vector boost. In some embodiments, the prime is a viral vector prime and the boost is a DNA or RNA (such as mRNA) boost.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Detection of Human Neoantigens

De-duplicated mapped reads for 11 SETD2-mutant and 21 SETD2-wt Renal Cell Carcinoma samples published (Simon et al., Genome Research, 2014, 24, 241-250) were imported into SeqMonk v1.48 (SeqMonk (RRID: SCR_001913), Babraham Institute, see, world wide web at “bioinformatics.babraham.ac.uk/projects/seqmonk/”). Reads were visualized against an annotated genome hg19. The presence of reads in the inter-genic regions suggested presence of DNA-contamination in RNA sequencing data. Percentage of reads mapped in gene regions were fluctuation between 50 to 75%, as was measured by SeqMonk. From NIH The database of Genotypes and Phenotypes (dbGaP) were obtained publicly available dataset of RNAseq data for dbGaP Study Accession: phs001287. Files for 10 SETD2-mutant and 34 SETD2-wt Renal Cell Carcinoma samples (number of sequencing files per sample fluctuated between 1 to 4, in total 110 files were used) were subset for subsequent analysis. In addition to publicly available data, RNA-sequensing for 10 SETD2-mutant and 34 SETD2-wt Renal Cell Carcinoma samples collected from patients who underwent treatment (Ilumina® Stranded Total RNA Prep, Ligation with Ribo-Zero Plus, 20040529, was used for library preparation, paeired reads, 150 bp long) was performed.

Reads were aligned to the human genome (GENCODE, hg38) containing intronic sequences. Transcript expression was quantified with eXpress following a published protocol (Pimentel et al., Nucleic Acid Res., 2016, 44, 838-851). KMA was implemented to identify genomic loci and level of intron retention (Smart et al., Nature Biotechnol., 2018, 36, 1056-1063). Intron was considered retained, if was detected by 5 unique reads; level of retention was in range 0 to 1, as ratue to expression of corresponding protein coding transcripts. Introns detected in more than 5 patients out of 10 SETD2 mutant samples in CPTAC3 or FCCC dataset, not detected in any control SETD2-wt specimens are the basis for the development of the therapeutic vaccine. Nucleotide sequences corresponding to the intronic regions and short fragments of the previous exonic sequences and the open reading frame were used to in silico translate peptide sequences derived from aberrantly retained introns into candidate neoantigen peptide sequences (Smart et al., Nature Biotechnol., 2018, 36, 1056-1063). This process resulted in 8675 short peptide sequences (SEQ ID NOs: 1-8675), which are the basis for the new vaccine for treatment of SETD2-mutant tumors.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A pharmaceutical composition comprising one or more peptides derived from aberrantly translated retained introns (ATaRIs).

2. The pharmaceutical composition according to claim 1, wherein the amino acid sequence of the ATaRI peptide comprises any one of SEQ ID NOs: 1-8675.

3. The pharmaceutical composition according to claim 1 or claim 2, comprising a plurality of peptides derived from ATaRIs.

4. The pharmaceutical composition according to any one of claims 1 to 3, further comprising one or more immune checkpoint inhibitors or one or more chemotherapeutic agents, or any combination thereof.

5. The pharmaceutical composition according to claim 4, wherein the immune checkpoint inhibitor comprises a PD-1 checkpoint inhibitor, a PD-L1 checkpoint inhibitor, a CTLA-4 checkpoint inhibitor, a TIGIT checkpoint inhibitor, or a LAG-3 checkpoint inhibitor, or any combination thereof.

6. The pharmaceutical composition according to claim 5, wherein the PD-1 checkpoint inhibitor comprises nivolumab, pembrolizumab, cetrelimab, or cemiplimab, or any combination thereof.

7. The pharmaceutical composition according to claim 5, wherein in the PD-L1 checkpoint inhibitor comprises atezolizumab, durvalab, or avelumab, or any combination thereof.

8. The pharmaceutical composition according to claim 5, wherein the CTLA-4 checkpoint inhibitor comprises ipilumumab or tremelimumab, or a combination thereof.

9. The pharmaceutical composition according to claim 4, wherein the chemotherapeutic agent comprises a tyrosine kinase inhibitor (TKI).

10. The pharmaceutical composition according to claim 9, wherein the TKI comprises bevacizumab, sunitinib, sorafenib, pazopanib, cabozantinib, lenvatinib, axitinib, or tivozanib, or any combination thereof.

11. The pharmaceutical composition according to claim 4, wherein the chemotherapeutic agent comprises a mammalian target of rapamycin (mTOR) inhibitor.

12. The pharmaceutical composition according to claim 11, wherein the mTOR inhibitor comprises temsirolimus or everolimus, or a combination thereof.

13. A method of treating a subject having cancer, wherein the subject comprises one or more deleterious mutations in the SET domain-containing 2 (SETD2) gene, the method comprising administering to the subject one or more peptides derived from aberrantly translated retained introns (ATaRIs) and/or one or more mRNA molecules encoding peptides derived from ATaRIs.

14. A method of immunizing a subject against cancer or eliciting an immune response to one or more peptides derived from aberrantly translated retained introns (ATaRIs) in a subject, wherein the subject comprises one or more deleterious mutations in the SET domain-containing 2 (SETD2) gene, the method comprising administering to the subject one or more peptides derived from ATaRIs and/or one or more mRNA molecules encoding peptides derived from ATaRIs.

15. The method according to claim 13 or claim 14, the method further comprising analyzing a biological sample obtained from the subject for the presence of the one or more deleterious mutations in the SETD2 gene.

16. The method according to any one of claims 13 to 15, wherein the amino acid sequence of the one or more peptides derived from ATaRI are selected from the group consisting of SEQ ID NOs: 1-8675.

17. The method according to any one of claims 13 to 16, wherein the cancer is SETD2-mutant liver cancer, mesothelioma, lung cancer, or kidney cancer.

18. The method according to claim 17, wherein the kidney cancer is clear cell renal cell carcinoma or papillary renal cell carcinoma.

19. The method according to any one of claims 13 to 18, wherein the biological sample is a tumor sample.

20. The method according to any one of claims 13 to 19, wherein the presence of the one or more deleterious mutations in the SETD2 gene is detected by nucleic acid sequencing or in-situ hybridization.

21. The method according to claim 20, wherein the nucleic acid sequencing is RNA sequencing.

22. The method according to any one of claims 13 to 21, wherein the deleterious mutations in the SETD2 gene lead to intron retention or activation of the unfolded protein response (UPR).

23. The method according to any one of claims 13 to 22, further comprising administering to the subject one or more immune checkpoint inhibitors or one or more chemotherapeutic agents, or any combination thereof.

24. The method according to claim 23, wherein the immune checkpoint inhibitor comprises a PD-1 checkpoint inhibitor, a PD-L1 checkpoint inhibitor, a CTLA-4 checkpoint inhibitor, a TIGIT checkpoint inhibitor, or a LAG-3 checkpoint inhibitor, or any combination thereof.

25. The method according to claim 24, wherein the PD-1 checkpoint inhibitor comprises nivolumab, pembrolizumab, cetrelimab, or cemiplimab, or any combination thereof.

26. The method according to claim 24, wherein in the PD-L1 checkpoint inhibitor comprises atezolizumab, durvalab, or avelumab, or any combination thereof.

27. The method according to claim 24, wherein the CTLA-4 checkpoint inhibitor comprises ipilumumab or tremelimumab, or a combination thereof.

28. The method according to claim 23, wherein the chemotherapeutic agent comprises a tyrosine kinase inhibitor (TKI).

29. The method according to claim 28, wherein the TKI comprises bevacizumab, sunitinib, sorafenib, pazopanib, cabozantinib, lenvatinib, axitinib, or tivozanib, or any combination thereof.

30. The method according to claim 23, wherein the chemotherapeutic agent comprises a mammalian target of rapamycin (mTOR) inhibitor.

31. The method according to claim 30, wherein the mTOR inhibitor comprises temsirolimus or everolimus, or a combination thereof.

32. The method according to any one of claims 13 to 31, further comprising administering another immunotherapy to the subject.

33. The method according to claim 32, wherein the another immunotherapy comprises chimeric antigen receptor-T cells (CAR-T), bone marrow transplant, adoptive transfer, interleukin-2, or interferon, or any combination thereof.
